(19) United States
(12) Patent Application Publication
(10) Pub. No.: US 2010/0280120 A1
(43) Pub. Date: Nov. 4, 2010

(54) COMPOUND INDUCING ANGIOGENESIS RESPONSE IN ISCHAEMIC TISSUES

(75) Inventor: Debatosh Datta, Kolkata (IN)

Correspondence Address: BINGHAM MCCUTCHEN LLP, 2020 K Street, N.W., Intellectual Property Department, WASHINGTON, DC, 20006, US

(73) Assignee: Green Cross Therapeutics Private Limited, Kolkata (IN)

(21) Appl. No.: 12/808,577

(22) PCT Filed: Dec. 15, 2008
(86) PCT No.: PCT/IN2008/000835

§ 371 Date: Jun. 16, 2010

(30) Foreign Application Priority Data
Dec. 17, 2007 (IN) 1689/KOL/2007

(51) Int. Cl. A61K 31/195; C07C 229/26

(52) U.S. Cl. 514/564; 562/561

(57) ABSTRACT

This invention deals with a novel straight chain compound, namely, 1,6-diamino alkanoic acid having a straight chain of six carbon atoms with two terminal amino groups and accompanying —COOH group, which is capable of effecting controlled formation of new blood vessels in ischaemic tissues, and their pharmaceutically acceptable salts and/or derivatives thereof. The compounds may be in laevo, dextro, activated laevo, activated dextro or oligomeric form. The invention also pertains to a process for preparing the aforesaid novel compound, which is illustrated by the accompanying drawing.

The present invention relates to novel straight chain compound capable of effecting angiogenesis in ischaemic tissues by controlled formation of new capillaries with the help of this low molecular weight compound, e.g. 1,6-diaminohexanoic acid. More particularly, the present invention pertains to effective utilization of a compound having a 6-membered straight chain structure having a plurality of amino groups on the carbon atoms at two extremities and carrying a carboxyl group on α-carbon atom beside an amino group, said amino groups being capable of forming electrostatic and/or hydrogen bonds under conditions of ischaemia following administration of said compound resulting in controlled angiogenesis.

Ischaemic tissues are the organs or parts of human body which on occasions or under special circumstances get no or less supply of blood, either temporarily or permanently, due to spasm, obstruction or narrowing of blood vessels. Such incidence of deprivation of blood supply could be due to formation of blood clots or thrombi obstructing the blood vessel, or else the reason could be metabolic. Either way, the process of obstruction is gradual, spanning over years, though the symptoms of obstructions are discernible and results could be devastating, leading even to mortality.

The most common instance of ischaemic tissue leading to severe discomfort or/and death is ischaemic cardiac tissue or myocardium. The process of obstruction of the coronary vessel(s) is more often metabolic in nature, known as “atherosclerosis”, where a particular type of lipids get deposited on the interior walls of coronary vessels restricting or constricting the effective diameter over the years. During this process of vessel-lumen narrowing, the affected part of myocardium gets progressively decreasing amounts of requisite gases and nutrients. When the narrowing process crosses a critical limit, the affected portion of myocardium dies in an acute circumstance known as ‘myocardial infarction’.

A similar instance affecting increasing number of people is cerebral stroke where cerebral/neural tissues get affected due to lack of blood supply to the affected region.

Although the conditions may sound or appear to be different or divergent from each other, they do have a common denominator, namely, ischaemia or lack of blood supply to the tissues of the affected part. A common answer to both types of afflictions narrated above appears to be the process of revascularization, where either the available vessels are recanalized by removal of plaque formed therein, bypassed or grafted (replaced). A more prudent and safe mode of treatment would be development of collateral vessels supplying the ischaemic tissues.

Present day mode of treating cerebral stroke resides in the tPA therapy followed by physiotherapy. Now-a-days it is suggested that the therapy should be initiated within the first one and a half hour post stroke episode. Obviously such initiation of therapy is virtually impossible under Indian conditions, particularly in the far flung rural areas, not to speak of the cost involvement. Such therapy is affordable and accessible to only a handful few living in metropolitan cities having improved healthcare facilities. Moreover such tPA therapy has recently been known to exhibit an undesirable side effect, namely, induced break through bleeding in about 15 to 20% cases after administration.

As pointed out earlier, development of vessels supplying the ischaemic tissues known as “angiogenesis” or “vasculogenesis” is likely to address a large number of clinical conditions. The present invention deals with a low-molecular weight, a 6-membered carbon chain (R-Group) containing compound having amino groups at two terminal carbon atoms, and a COOH group on α-carbon atom having the capacity to form charged centres on N-atoms, which in turn bond with the charges residing on the receptor protein and angiogenic factor and/or growth factor. It has been observed that there is an abundance of H⁺-ions in and around ischaemic tissues, in comparison with non-ischaemic tissues and these cause protonation of terminal amino groups of the compound, forming an ionized NH₃⁺-centre at the two ends, which in turn form electrostatic bonds with the negative charges residing both on the receptor protein (RP) present in the endothelial cells membrane on the one hand and angiogenic factors on the other. The latter, namely, angiogenic factor or growth factor (AF/GF) acts as a ligand, establishing a link with the receptor by utilizing the charged terminals of the subject compound.

The angiogenic factors referred to above are known to be liberated in excess by the endothelial cells in the ischaemic tissue. These factors (AFs) have their receptors (a kind of protein) in the endothelial cell membrane. Establishment of a bridge by the protonated hexanoic acid moiety brings about an activation of capillaries with consequent angiogenesis through endothelial cell division and migration.

The foregoing compound helps in induction of new capillary formation in ischaemic tissue reperfusion in following instances: e.g. for example (non-exhaustive):
(a) ischaemic cerebral/neural tissue(s) and as a vaccine for prevention of cerebral/neural tissue ischaemia,
(b) ischaemic myocardium/cardiac tissue(s) and as a vaccine/agent for prevention of cardiac tissue ischaemia (angina),
(c) in ischaemic diabetic vasculopathy as in diabetic microangiopathy in diabetic bone marrow, diabetic microangiopathy in nephrons, diabetic wounds and ulcers and the like, and as an agent for prevention of diabetic microangiopathy in any anatomic location(s),
(d) in enhancing bioavailability of chemotherapeutic agent(s) and/or radio sensitizing agent(s) in case of ischaemic tumor tissue(s) through/during chemotherapy and/or radiotherapy, thereby enhancing efficacy of CT/RT, and
(e) in induction of enhanced angiogenic response in reperfusion of various other ischaemic tissues like ischaemic renal tissue in ARF and CRF, ischaemic limb, ischaemic placental tissue, etc., and as a preventive agent against development of ischaemia in above tissues.

The activation of the 1,6-diamino-hexanoic acid, with consequent establishment of a bridge between RP (receptor protein) and AF/GF (angiogenic factor/growth factor) may be represented as follows:

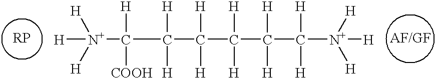

The native compound as in FIG. 2 gets activated by a process of protonation because of excess proton build-up in ischaemic tissues which triggers the change in conformation of the diaminohexanoic acid moiety, and enabling it to fit snugly between RP and AF/GF. Once a bridge is established, concerned cellular division and differentiation is augmented through a “more stable” receptor (RP)—ligand (AF/GF) complex on the endothelial cell membrane. This postulated “augmentation of receptor-ligand complex” is quite akin to enzyme-substrate interaction through the mediation of co-factors, and is not described in current biology and probably raises the possibility of a new concept in modern biology—“co-factor in receptor-ligand bridging/binding”. Formation of the aforesaid activated compound has been shown in FIG. 2 of the drawings.

The main object of this invention is to bring about angiogenesis in ischaemic tissues by employing a low molecular weight compound like 1,6-diamino-hexa-1-noic acid. This compound gets protonated and consequently activated in the ischaemic (tissue) bed (e.g. wound in acute and chronic conditions) thereby acting as a “micro-sensor”/“molecular sensor”, and the activated molecule bridges the AFs to their receptors, thereby accelerating the process of healing of the ischaemic (injured/wounded) tissue bed. Such low molecular weight compounds open up completely new regime of wound/ischaemia management without any manifested side effects or contra-indications.

A further object of this is to provide a novel diamino compound, namely, 1,6-diamino-hexa-1-noic acid, a straight chain aliphatic saturated compound having two terminal amino groups and an accompanying —COOH group, an pharmaceutically acceptable salts and/or derivatives thereof.

A still further object of this invention is to provide a process for preparing the aforesaid novel compound having two terminal amino groups and a —COOH group.

In accordance with this invention, there is provided a new compound, namely, 1,6-diamino-hexa-i-noic acid capable of bringing about angiogenesis in ischaemic tissues and pharmaceutically acceptable salts and/or derivatives thereof, including inter alia, an L-, D-, an activated L-, an activated D- or an oligomer or mixtures thereof.

The present invention also relates to a process for preparing the aforementioned novel diamino alkanoic acid compound and pharmaceutically acceptable salts and/or derivatives thereof, characterized in that the said preparation is carried out in accordance with the sequence of reactions shown in the accompanying drawing wherein the undernoted abbreviations have been used:

Cbz-Cl: Benzyloxy carbonyl chloride

### THF: Tetrahydrofuran

PTSA: p-Toluene sulfonic acid
BH₃ DMS: Borane dimethylsulfide
PCC: Pyridinium chlorochromate

### DCM: Dichloromethane and

(CH₂O)ₙ: Para formaldehyde

The following examples are given below based on the tests conducted, which are given by way of illustration and not by way of limitation.

### EXAMPLE-1

### Studies on the Animal Model to Ascertain the Toxicity of the Novel Compound

i. Animal Model:

All animal experiments were performed following ‘Principles of laboratory animal care’ (NIH publication No. 85-23, revised in 1985) as well as specific Indian laws on ‘Protection of Animals’ under the provision o authorized investigators. Swiss albino mice (˜20 g each; 10-12 weeks old; 3 mice in each group) were randomly divided into (i) untreated set (i.v. injected with PBS as vehicle only) and (ii) drug-treated set (Compound of this invention 0.1, 0.3, 1.0, 3.0, 10 and 30 mg/kg body weight) for 14 days. Untreated mice received sterile PBS as carrier vehicle. Doxorubicin (5 mg/kg body weight) was used as positive control for toxicity assays.

ii. Statistical Analysis

For statistical analysis, one-way analysis of variance (ANOVA) was conducted, followed by the Newman-Keuls multiple comparison test. Mean differences with p<0.05 were considered statistically significant.

iii. Systemic Toxicity

Hepato and cardio toxicity due to drug regimens are the shortfall in many instances. The serum levels of GOT, GPT and ALP are clinical indicators of drug-induced toxicity. Blood samples were collected from the retro-orbital plexus of normal and the subject compound-treated mice. Total serum glutamate oxaloacetate transaminase (SGPT), glutamate pyruvate transaminase (SGPT), and alkaline phosphate were assayed according to standard protocols. In the aforesaid mice model, the levels of serum GOT and GPT were significantly increased in doxorubicin-treated mice (p<0.05) which served as a positive control for toxicity assays. Compound of this invention at doses between 0.1 mg/kg body weight and 30 mg/kg body weight showed no significant toxicity.

iv. Effect of Compound on Serum Toxicity Marker Enzymes in Mice

Serum GOT, GPT and ALP activities as a marker of systemic toxicity were measured from blood of untreated or treated mice. Doxorubicin treatment increased SGPT to 68 IU/dL; SGOT to 79 IU/dL, and ALP to 43 KA units. The data collected agreed well with the three independent sets of experiments done individually.

### EXAMPLE-2

### i. Screening of Different Cells

To study the toxic effects of compound, a wide spectrum of primary cells or cell lines from various origin were screened for viability, using Trypan blue-exclusion test. Cells were grown in cultures in DMEM/F-12 (1:1) or RPMI 1640 media supplemented with 10% FBS, insulin (0.1 units/ml), L-glutamine (2 mM), sodium pyruvate (100 μg/ml), non-essential amino acids (100 μM), penicillin (100 units/ml) and streptomycin (100 μg/μl). Cells were incubated at 37° C. in a humidified atmosphere of 5% CO₂. Data collected showed that compound at doses 12.5 to 200 μg/mL range, showed no toxic effects (as the percent of cell killing was less than 5% up to 50 μg/mL doses which is 50 times more than the effective dose). In 100 and 200 μg/mL doses the drug showed mild toxicity which is even below the significant killing.

### ii. Effect of the Compound on In Vitro Toxicity on Human Cell Lines

Different cells from different origins were cultured in vitro. Various doses of GC-S were added in culture medium. After 24 h number of surviving cells was counted. Each experiment was performed in triplicate and the values agreed well with those of independent experiments

### EXAMPLE-3

### To Test Angiogenic Potential of Compound

The angiogenic potential of compound was tested by binding human VEGF to its putative receptors. Human lung epithelial cells (A549 which are highly metastatic and therefore angiogenic in nature) were grown on coverslip in DMEM/F-12 (1:1) or RPMI 1640 media supplemented with 10% FBS, insulin (0.1 units/ml), L-glutamine (2 mM), sodium pyruvate (100 μg/ml), non-essential amino acids (100 μM), penicillin (100 units/ml) and streptomycin (100 μg/μl). Cells were incubated at 37° C. in a humidified atmosphere of 5% CO₂. The pH of the medium was maintained at 6.2 for the binding assay of VEGF to its receptor in the presence of compound. The cells were pre-treated with media alone or with 25 μg/ml compound for 30 min. The cells were then incubated with 10 ng/ml human VEGF for further 30 min at 37° C. in a humidified atmosphere of 5% CO₂. The cells were fixed and stained with anti-VEGF antibody coupled with Alexaflour 488. The cells were visualized in Zeiss-confocal microscope.

### Results

The results showed that normal A549 cell has low VEGF binding on the membrane, which remains unaltered after compound treatment. Interestingly when cells were pre-incubated with compound, the binding of VEGF to its receptor increased 10-15 times or even higher.

While the invention has been described in detail and with reference to specific embodiments thereof, it will be apparent to one skilled in the art that various changes and modifications can be made therein without deviating or departing from the spirit and scope of the invention. Thus the disclosure contained herein includes within its ambit the various equivalents and substitutes as well.

Having described the invention in detail with particular reference to the illustrative examples given above and also to the accompanying drawing, it will be more specifically defined by claims appended hereafter.

1-4. (canceled)

5. A compound having the following formula

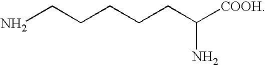

6. A pharmaceutically acceptable salt of the compound of claim 5.

7. A compound according to claim 5, which is in levo, dextro, activated levo, activated dextro or oligomeric form.

8. The compound according to claim 7, which is in levo form.

9. The compound according to claim 7, which is in dextro form.

10. The compound according to claim 7, which is in activated levo form.

11. The compound according to claim 7, which is in activated dextro form.

12. The compound according to claim 7, which is in oligomeric form.

13. A composition comprising the compound of claim 5 and a carrier therefor.

14. A composition comprising the pharmaceutically acceptable salt of claim 6 and a pharmaceutically acceptable carrier therefor.

15. A method comprising the step of administering the composition of claim 14 to a mammal, thereby inducing capillary formation in ischaemic tissue reperfusion.

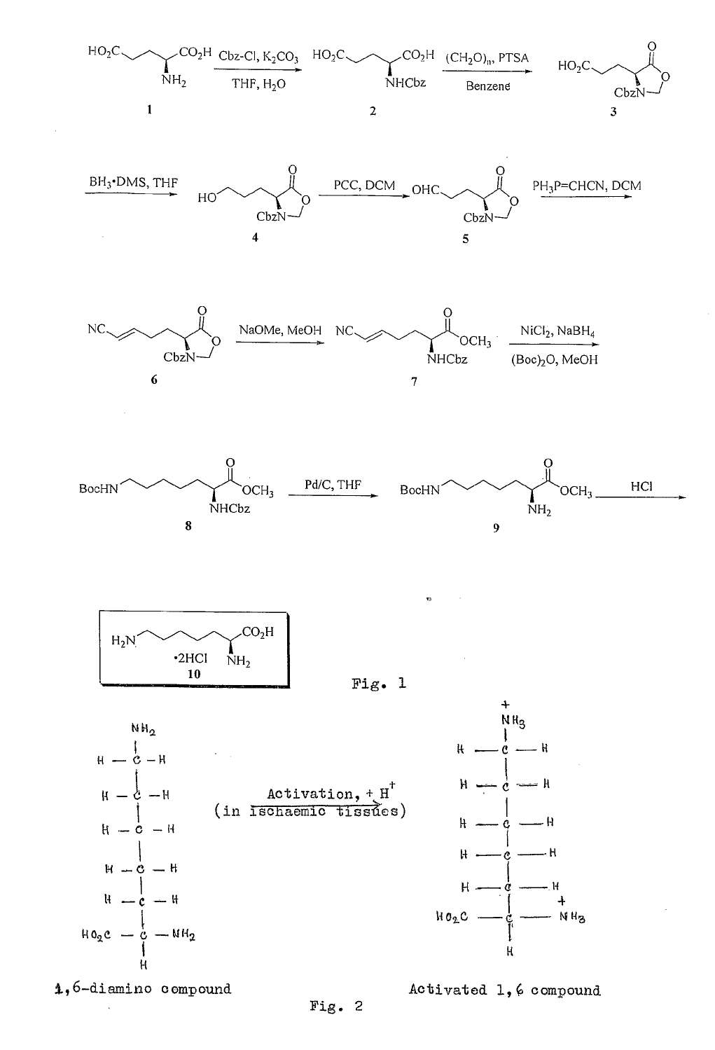

16. A method of making a compound according to claim 5, comprising the steps shown in FIG. 1.
